(19) United States
(12) Patent Application Publication
(10) Pub. No.: US 2010/0171108 A1
(43) Pub. Date: Jul. 8, 2010

(54) USE OF N,N'-BIS(1,1-DIHYDROPERFLUORO-C3-C5-ALKYL)-PERYLENE-3,4:9,10- TETRACARBOXYLIC DIIMIDES

(75) Inventors: Martin Koenemann, Mannheim (DE); Frank Wuerthner, Hoechberg (DE); Ruediger Schmidt, Paderborn (DE); Zhenan Bao, Stanford, CA (US); Joon Hak Oh, Palo Alto, CA (US)

Correspondence Address: OBLON, SPIVAK, MCCLELLAND MAIER & NEUSTADT, L.L.P., 1940 DUKE STREET, ALEXANDRIA, VA, 22314, US

(73) Assignees: BASF SE, Ludwigshafen (DE); The Board of Trust. of the Lelnd Stanf. Jun. Univ., Palo Alto, CA (US)

(21) Appl. No.: 12/666,127

(22) PCT Filed: Jun. 20, 2008
(86) PCT No.: PCT/EP08/57829

§ 371 Date: Dec. 22, 2009

Related U.S. Application Data
(60) Provisional application No. 60/945,704, filed on Jun. 22, 2007.

(51) Int. Cl. H01L 51/30; H01L 51/54; H01L 51/56; H01L 51/40; C07D 471/02

(52) U.S. Cl. 257/40; 438/99; 546/37; 257/E51.025; 438/478; 257/E51.026; 257/E51.041

(57) ABSTRACT

The present invention relates to the use of N,N′-bis(1,1-dihydroperfluoro-C₃-C₅-alkyl)perylene-3,4:9,10-tetracarboxylic diimides as charge transport materials or exciton transport materials.

The present invention relates to the use of N,N′-bis(1,1-dihydroperfluoro-C₃-C₅-alkyl)perylene-3,4:9,10-tetracarboxylic diimides as charge transport materials or exciton transport materials.

It is expected that, in the future, not only the classical inorganic semiconductors but increasingly also organic semiconductors based on low molecular weight or polymeric materials will be used in many sectors of the electronics industry. In many cases, these organic semiconductors have advantages over the classical inorganic semiconductors, for example better substrate compatibility and better processibility of the semiconductor components based on them. They allow processing on flexible substrates and enable their interface orbital energies to be adjusted precisely to the particular application range by the methods of molecular modeling. The significantly reduced costs of such components have brought a renaissance to the field of research of organic electronics. Organic electronics is concerned principally with the development of new materials and manufacturing processes for the production of electronic components based on organic semiconductor layers. These include in particular organic field-effect transistors (OFETs) and organic light-emitting diodes (OLEDs), and photovoltaics. Great potential for development is ascribed to organic field-effect transistors, for example in storage elements and integrated optoelectronic devices. Organic light-emitting diodes (OLEDs) utilize the property of materials of emitting light when they are excited by electrical current. OLEDs are especially of interest as an alternative to cathode ray tubes and liquid-crystal displays for producing flat visual display units. Owing to the very compact design and the intrinsically low power consumption, devices which comprise OLEDs are suitable especially for mobile applications, for example for applications in cell phones, laptops, etc. A great potential for development is also ascribed to materials which have maximum transport widths and high mobilities for light-induced excited states (high exciton diffusion lengths) and are thus advantageously suitable for use as an active material in so-called excitonic solar cells. It is generally possible with solar cells based on such materials to achieve very good quantum yields.

There is therefore a great need for organic compounds which are suitable as charge transport materials or exciton transport materials.

P. R. L. Malenfant et al. describe, in Applied Physics Letters Vol. 80, No. 14 (2002), p. 2517-2519, organic field-effect transistors based on N,N′-dioctyl-3,4:9,10-perylenetetracarboximide.

K. Deyama et al. describe, in Dyes and Pigments, Vol. 30, No. 1, p. 73-78, 1996, 3,4:9,10-perylenetetracarboximides in which the imide nitrogen atoms bear perfluoroalkyl radicals including n-heptafluoropropyl. Their use as semiconductors in organic field-effect transistors (OFTs) and in organic photovoltaics (OPVs) is not described.

Min-Min Shi et al. describe, in Acta Chimica Sinica, Vol. 64, 2006, No. 8, p. 721-726, the electron mobilities of N,N′-bisperfluorophenyl-3,4:9,10-perylenetetracarboximide and N,N′-bis(1,1-dihydroperfluorooctyl)-3,4:9,10-perylenetetracarboximide. The electron mobilities of these compounds are still in need of improvement with regard to use as organic field-effect transistors and in organic photovoltaics. A possible use in excitonic solar cells is not described.

Z. Bao et al. describe, in Chem. Mater. 2007, 19, 816-824, the use of fluorinated derivatives of perylenediimides as n-semiconductors in thin-film transistors (TFTs). In this case, perylenediimides in which the imide nitrogen atoms bear fluorinated aryl radicals are used.

PCT/EP 2006/070143 (=WO2007/074137), which was unpublished at the priority date of this application, describes compounds of the general formula (A)

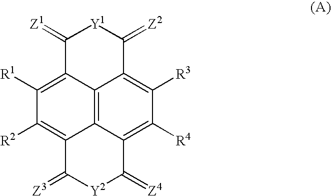

where
at least one of the R¹, R², R³ and R⁴ radicals is a substituent which is selected from Br, F and CN,
Y¹ is O or NR^{a} where R^{a} is hydrogen or an organyl radical,
Y² is O or NR^{b} where R^{b} is hydrogen or an organyl radical,
Z¹ and Z² are each independently O or NR^{c} where R^{c} is an organyl radical,
Z³ and Z⁴ are each independently O or NR^{d} where R^{d} is an organyl radical,
where, in the case that Y¹ is NR^{a} and at least one of the Z¹ and Z² radicals is NR^{c}, R^{a} with one R^{c} radical may also together be a bridging group having from 2 to 5 atoms between the flanking bonds, and
where, in the case that Y² is NR^{b} and at least one of the Z³ and Z⁴ radicals is NR^{d}, R^{b} with one R^{d} radical may also together be a bridging group having from 2 to 5 atoms between the flanking bonds,
and their use as n-semiconductors in organic field-effect transistors.

PCT/EP 2007/051532 (=WO 2007/093643), which was unpublished at the priority date of the present application, describes the use of compounds of the general formula (B)

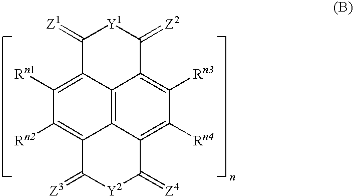

where
n is 2, 3 or 4,
at least one of the Rⁿ¹, Rⁿ², Rⁿ³ and Rⁿ⁴ radicals is fluorine,
optionally at least one further Rⁿ¹, Rⁿ², Rⁿ³ and Rⁿ⁴ radical is a substituent which is selected independently from Cl and Br, and the remaining radicals are each hydrogen,
Y¹ is O or NR^{a} where R^{a} is hydrogen or an organyl radical,
Y² is O or NR^{b} where R^{b} is hydrogen or an organyl radical,
Z¹, Z², Z³ and Z⁴ are each O,
where, in the case that Y¹ is NR^{a}, one of the Z¹ and Z² radicals may also be NR^{c}, where the R^{a} and R^{c} radicals together are a bridging group having from 2 to 5 atoms between the flanking bonds, and
where, in the case that Y² is NR^{b}, one of the Z³ and Z⁴ radicals may also be NR^{d}, where the R^{b} and R^{d} radicals together are a bridging group having from 2 to 5 atoms between the flanking bonds,
as semiconductors, especially n-semiconductors, in organic electronics, especially for organic field-effect transistors, solar cells and organic light-emitting diodes.

U.S. Pat. No. 7,026,643 likewise describes the use of N,N′-3,4:9,10-perylenetetracarboximides as a semiconductor material for organic thin-film transistors, and specifically N,N′-di(n-1H,1H-perfluorooctyl)perylene-3,4:9,10-tetracarboximide is used.

It has now been found that, surprisingly, N,N′-bis(1,1-dihydroperfluoro-C₃-C₅-alkyl)perylene-3,4:9,10-tetracarboxylic diimides are suitable particularly advantageously as charge transport materials or exciton transport materials. They are notable especially as air-stable n-semiconductors with exceptionally high charge mobilities.

The invention therefore firstly provides for the use of compounds of the general formula (I)

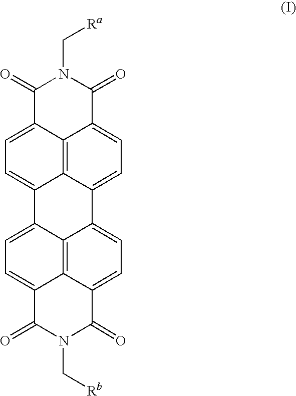

where
R^{a} and R^{b} are each independently perfluoro-C₂-C₄-alkyl,
as charge transport materials or exciton transport materials.

In the compounds of the formula (I), R^{a} and R^{b} radicals may have identical or different definitions. In a preferred embodiment, the R^{a} and R^{b} radicals have identical definitions.

R^{a} and R^{b} are preferably each independently selected from pentafluoroethyl (C₂F₅), n-heptafluoropropyl (n-C₃F₇), heptafluoroisopropyl (CF(CF₃)₂), n-nonafluorobutyl (n-C₄F₉), and also C(CF₃)₃, CF₂CF(CF₃)₂, CF(CF₃)(C₂F₅).

R^{a} and R^{b} are preferably each n-heptafluoropropyl (n-C₃F₇).

The compounds of the formula (I) are particularly advantageously suitable as organic semiconductors. They generally function as n-semiconductors. When the compounds of the formula (I) used in accordance with the invention are combined with other semiconductors and the position of the energy levels results in the other semiconductors functioning as n-semiconductors, the compounds (I) may also function as p-semiconductors in exceptional cases.

The compounds of the formula (I) are notable for their air stability. Moreover, they have a high charge transport mobility which clearly sets them apart from known organic semiconductor materials. They additionally have a high on/off ratio.

The compounds of the formula (I) are particularly advantageously suitable for organic field-effect transistors. They may be used, for example, for the production of integrated circuits (ICs), for which customary n-channel MOSFETs (metal oxide semiconductor field-effect transistors) have been used to date. These are then CMOS-like semiconductor units, for example for microprocessors, microcontrollers, static RAM and other digital logic circuits. For the production of semiconductor materials, the compounds of the formula (I) can be processed further by one of the following processes: printing (offset, flexographic, gravure, screenprinting, inkjet, electrophotography), evaporation, laser transfer, photolithography, drop-casting. They are especially suitable for use in displays (specifically large-surface area and/or flexible displays) and RFID tags.

The compounds of the formula (I) are particularly advantageously suitable as electron conductors in organic field-effect transistors, organic solar cells and in organic light-emitting diodes. They are also particularly advantageous as an exciton transport material in excitonic solar cells.

The compounds of the formula (I) are also particularly advantageously suitable as fluorescent dyes in a display based on fluorescence conversion. Such displays comprise generally a transparent substrate, a fluorescent dye present on the substrate and a radiation source. Typical radiation sources emit blue (color by blue) or UV light (color by uv). The dyes absorb either the blue or the UV light and are used as green emitters. In these displays, for example, the red light is generated by exciting the red emitter by means of a green emitter which absorbs blue or UV light. Suitable color-by-blue displays are described, for example, in WO 98/28946. Suitable color-by-UV displays are described, for example, by W. A. Crossland, I. D. Sprigle and A. B. Davey in Photoluminescent LCDs (PL-LCD) using phosphors, Cambridge University and Screen Technology Ltd., Cambridge, UK. The compounds of the formula (I) are also particularly suitable in displays which, based on an electrophoretic effect, switch colors on and off via charged pigment dyes. Such electrophoretic displays are described, for example, in US 2004/0130776.

The compounds of the formula (I) are also particularly suitable for laser welding or for heat management.

The invention further provides organic field-effect transistors comprising a substrate with at least one gate structure, a source electrode and a drain electrode, and at least one compound of the formula (I) as defined above as a semiconductor, especially as an n-semiconductor.

The invention further provides substrates having a plurality of organic field-effect transistors, wherein at least some of the field-effect transistors comprise at least one compound of the formula (I) as defined above.

The invention also provides semiconductor units which comprise at least one such substrate.

A specific embodiment is a substrate with a pattern (topography) of organic field-effect transistors, each transistor comprising
an organic semiconductor disposed on the substrate;
a gate structure for controlling the conductivity of the conductive channel; and
conductive source and drain electrodes at the two ends of the channel,
   the organic semiconductor consisting of at least one compound of the formula (I) or comprising a compound of the formula (I). In addition, the organic field-effect transistor generally comprises a dielectric.

A further specific embodiment is a substrate having a pattern of organic field-effect transistors, each transistor forming an integrated circuit or being part of an integrated circuit and at least some of the transistors comprising at least one compound of the formula (I).

Suitable substrates are in principle the materials known for this purpose. Suitable substrates comprise, for example, metals (preferably metals of groups 8, 9, 10 or 11 of the Periodic Table, such as Au, Ag, Cu), oxidic materials (such as glass, ceramics, SiO₂, especially quartz), semiconductors (e.g. doped Si, doped Ge), metal alloys (for example based on Au, Ag, Cu, etc.), semiconductor alloys, polymers (e.g. polyvinyl chloride, polyolefins such as polyethylene and polypropylene, polyesters, fluoropolymers, polyamides, polyimides, polyurethanes, polyalkyl (meth)acrylates, polystyrene and mixtures and composites thereof), inorganic solids (e.g. ammonium chloride), paper and combinations thereof. The substrates may be flexible or inflexible, and have a curved or planar geometry, depending on the desired use.

A typical substrate for semiconductor units comprises a matrix (for example a quartz or polymer matrix) and, optionally, a dielectric top layer.

Suitable dielectrics are SiO₂, polystyrene, poly-α-methylstyrene, polyolefins (such as polypropylene, polyethylene, polyisobutene), polyvinylcarbazole, fluorinated polymers (e.g. Cytop), cyanopullulans (e.g. CYMM), polyvinylphenol, poly-p-xylene, polyvinyl chloride, or polymers crosslinkable thermally or by atmospheric moisture. Specific dielectrics are “self-assembled nanodielectrics”, i.e. polymers which are obtained from monomers comprising SiCl functionalities, for example Cl₃SiOSiCl₃, Cl₃Si—(CH₂)₆—SiCl₃, Cl₃Si—(CH₂)₁₂—SiCl₃, and/or which are crosslinked by atmospheric moisture or by addition of water diluted with solvents (see, for example, Faccietti Adv. Mat. 2005, 17, 1705-1725). Instead of water, it is also possible for hydroxyl-containing polymers such as polyvinylphenol or polyvinyl alcohol or copolymers of vinylphenol and styrene to serve as crosslinking components. It is also possible for at least one further polymer to be present during the crosslinking operation, for example polystyrene, which is then also crosslinked (see Facietti, US patent application 2006/0202195).

The substrate may additionally have electrodes, such as gate, drain and source electrodes of OFETs, which are normally localized on the substrate (for example deposited onto or embedded into an nonconductive layer on the dielectric). The substrate may additionally comprise conductive gate electrodes of the OFETs, which are typically arranged below the dielectric top layer (i.e. the gate dielectric).

In a specific embodiment, an insulator layer (gate insulating layer) is present on at least part of the substrate surface. The insulator layer comprises at least one insulator which is preferably selected from inorganic insulators such as SiO₂, silicon nitride (Si₃N₄), etc., ferroelectric insulators such as Al₂O₃, Ta₂O₅, La₂O₅, TiO₂, Y₂O₃, etc., organic insulators such as polyimides, benzocyclobutene (BCB), polyvinyl alcohols, polyacrylates, etc., and combinations thereof.

Suitable materials for source and drain electrodes are in principle electrically conductive materials. These include metals, preferably metals of groups 6, 7, 8, 9, 10 or 11 of the Periodic Table, such as Pd, Au, Ag, Cu, Al, Ni, Cr, etc. Also suitable are conductive polymers such as PEDOT (=poly(3,4-ethylenedioxythiophene)):PSS (=poly(styrenesulfonate)), polyaniline, surface-modified gold, etc. Preferred electrically conductive materials have a specific resistance of less than 10⁻³ ohm×meter, preferably less than 10⁻⁴ ohm×meter, especially less than 10⁻⁶ or 10⁻⁷ ohm×meter.

In a specific embodiment, drain and source electrodes are present at least partly on the organic semiconductor material. It will be appreciated that the substrate may comprise further components as used customarily in semiconductor materials or ICs, such as insulators, resistors, capacitors, conductor tracks, etc.

The electrodes may be applied by customary processes, such as evaporation, lithographic processes or another structuring process.

The semiconductor materials may also be processed with suitable auxiliaries (polymers, surfactants) in disperse phase by printing.

In a first preferred embodiment, the deposition of at least one compound of the general formula (I) (and if appropriate further semiconductor materials) is carried out by a gas phase deposition process (physical vapor deposition, PVD). PVD processes are performed under high-vacuum conditions and comprise the following steps: evaporation, transport, deposition. It has been found that, surprisingly, the compounds of the general formula (I) are suitable particularly advantageously for use in a PVD process, since they essentially do not decompose and/or form undesired by-products. The material deposited is obtained in high purity. In a specific embodiment, the deposited material is obtained in the form of crystals or comprises a high crystalline content. In general, for the PVD, at least one compound of the general formula (I) is heated to a temperature above its evaporation temperature and deposited on a substrate by cooling below the crystallization temperature. The temperature of the substrate in the deposition is preferably within a range from about 20 to 250° C., more preferably from 50 to 200° C. It has been found that, surprisingly, elevated substrate temperatures in the deposition of the compounds of the formula (I) can have advantageous effects on the properties of the semiconductor elements achieved.

The resulting semiconductor layers generally have a thickness which is sufficient for ohmic contact between source and drain electrodes. The deposition can be effected under an inert atmosphere, for example, under nitrogen, argon or helium.

The deposition is effected typically at ambient pressure or under reduced pressure. A suitable pressure range is from about 10⁻⁷ to 1.5 bar.

The compound of the formula (I) is preferably deposited on the substrate in a thickness of from 10 to 1000 nm, more preferably from 15 to 250 nm. In a specific embodiment, the compound of the formula (I) is deposited at least partly in crystalline form. For this purpose, especially the above-described PVD process is suitable. Moreover, it is possible to use previously prepared organic semiconductor crystals. Suitable processes for obtaining such crystals are described by R. A. Laudise et al. in “Physical Vapor Growth of Organic Semi-Conductors”, Journal of Crystal Growth 187 (1998), pages 449-454, and in “Physical Vapor Growth of Centimeter-sized Crystals of α-Hexathiophene”, Journal of Crystal Growth 1982 (1997), pages 416-427, which are incorporated here by reference.

In a second preferred embodiment, the deposition of at least one compound of the general formula (I) (and if appropriate further semiconductor materials) is effected by spin-coating. Surprisingly, it is thus also possible to use the compounds of the formula (I) used in accordance with the invention in a wet processing method to produce semiconductor substrates. The compounds of the formula (I) should thus also be suitable for producing semiconductor elements, especially OFETs or based on OFETs, by a printing process. It is possible for this purpose to use customary printing processes (inkjet, flexographic, offset, gravure; intaglio printing, nanoprinting). Preferred solvents for the use of compounds of the formula (I) in a printing process are aromatic solvents such as toluene, xylene, etc. It is also possible to add thickening substances such as polymers, for example polystyrene, etc., to these “semiconductor inks”. In this case, the dielectrics used are the aforementioned compounds.

In a preferred embodiment, the inventive field-effect transistor is a thin-film transistor (TFT). In a customary construction, a thin-film transistor has a gate electrode disposed on the substrate, a gate insulation layer disposed thereon and on the substrate, a semiconductor layer disposed on the gate insulator layer, an ohmic contact layer on the semiconductor layer, and a source electrode and a drain electrode on the ohmic contact layer.

In a preferred embodiment, the surface of the substrate, before the deposition of at least one compound of the general formula (I) (and if appropriate of at least one further semiconductor material), is subjected to a modification. This modification serves to form regions which bind the semiconductor materials and/or regions on which no semiconductor materials can be deposited. The surface of the substrate is preferably modified with at least one compound (C1) which is suitable for binding to the surface of the substrate and to the compounds of the formula (I). In a suitable embodiment, a portion of the surface or the complete surface of the substrate is coated with at least one compound (C1) in order to enable improved deposition of at least one compound of the general formula (I) (and if appropriate further semiconductive compounds). A further embodiment comprises the deposition of a pattern of compounds of the general formula (C1) on the substrate by a corresponding production process. These include the mask processes known for this purpose and so-called “patterning” processes, as described, for example, in U.S. Ser. No. 11/353,934, which is incorporated here fully by reference.

Suitable compounds of the formula (C1) are capable of a binding interaction both with the substrate and with at least one semiconductor compound of the general formula (I). The term “binding interaction” comprises the formation of a chemical bond (covalent bond), ionic bond, coordinative interaction, van der Waals interactions, e.g. dipole-dipole interactions etc.), and combinations thereof. Suitable compounds of the general formula (C1) are:
silane, phosphonic acids, carboxylic acids, hydroxamic acids, such as alkyltrichlorosilanes, e.g. n-octadecyltrichlorosilane; compounds with trialkoxysilane groups, e.g. alkyltrialkoxysilanes such as n-octadecyltrimethoxysilane, n-octadecyltriethoxysilane, n-octadecyltri(n-propyl)oxysilane, n-octadecyltri(isopropyl)oxysilane; trialkoxyaminoalkylsilanes such as triethoxyaminopropylsilane and N[(3-triethoxysilyl)propyl]ethylenediamine; trialkoxyalkyl 3-glycidyl ether silanes such as triethoxypropyl 3-glycidyl ether silane; trialkoxyallylsilanes such as allyltrimethoxysilane; trialkoxy(isocyanatoalkyl)silanes; trialkoxysilyl(meth)acryloyloxyalkanes and trialkoxysilyl(meth)acrylamidoalkanes such as 1-triethoxysilyl-3-acryl-oyl-oxypropane.
amines, phosphines and sulfur-comprising compounds, especially thiols.

The compound (C1) is preferably selected from alkyltrialkoxysilanes, especially n-octadecyltrimethoxysilane, n-octadecyltriethoxysilane; hexaalkyldisilazanes, and especially hexamethyldisilazane (HMDS); C₈-C₃₀-alkylthiols, especially hexadecanethiol; mercaptocarboxylic acids and mercaptosulfonic acids, especially mercaptoacetic acid, 3-mercaptopropionic acid, mercaptosuccinic acid, 3-mercapto-1-propanesulfonic acid and the alkali metal and ammonium salts thereof.

Various semiconductor architectures comprising the inventive semiconductors are also conceivable, for example top contact, top gate, bottom contact, bottom gate, or else a vertical construction, for example a VOFET (vertical organic field-effect transistor), as described, for example, in US 2004/0046182.

The layer thicknesses are, for example, from 10 nm to 5 μm in semiconductors, from 50 nm to 10 μm in the dielectric; the electrodes may, for example, be from 20 nm to 1 μM. The OFETs may also be combined to form other components such as ring oscillators or inverters.

A further aspect of the invention is the provision of electronic components which comprise a plurality of semiconductor components, which may be n- and/or p-semiconductors. Examples of such components are field-effect transistors (FETs), bipolar junction transistors (BJTs), tunnel diodes, converters, light-emitting components, biological and chemical detectors or sensors, temperature-dependent detectors, photodetectors such as polarization-sensitive photodetectors, gates, AND, NAND, NOT, OR, TOR and NOR gates, registers, switches, timer units, static or dynamic stores and other dynamic or sequential, logical or other digital components including programmable switches.

A specific semiconductor element is an inverter. In digital logic, the inverter is a gate which inverts an input signal. The inverter is also referred to as a NOT gate. Real inverter switches have an output current which constitutes the opposite of the input current. Typical values are, for example, (0, +5V) for TTL switches. The performance of a digital inverter reproduces the voltage transfer curve (VTC), i.e. the plot of input current against output current. Ideally, it is a staged function and, the closer the real measured curve approximates to such a stage, the better the inverter is. In a specific embodiment of the invention, the compounds of the formula (I) are used as organic n-semiconductors in an inverter.

The compounds of the formula (I) are also particularly advantageously suitable for use in organic photovoltaics (OPVs). In principle, these compounds are suitable for use in dye-sensitized solar cells. However, preference is given to their use in solar cells which are characterized by diffusion of excited states (exciton diffusion). In this case, one or both of the semiconductor materials utilized is notable for a diffusion of excited states (exciton mobility). Also suitable is the combination of at least one semiconductor material which is characterized by diffusion of excited states with polymers which permit conduction of the excited states along the polymer chain. In the context of the invention, such solar cells are referred to as excitonic solar cells. The direct conversion of solar energy to electrical energy in solar cells is based on the internal photo effect of a semiconductor material, i.e. the generation of electron-hole pairs by absorption of photons and the separation of the negative and positive charge carriers at a p-n transition or a Schottky contact. An exciton can form, for example, when a photon penetrates into a semiconductor and excites an electron to transfer from the valence band into the conduction band. In order to generate current, the excited state generated by the absorbed photons must, however, reach a p-n transition in order to generate a hole and an electron which then flow to the anode and cathode. The photovoltage thus generated can bring about a photocurrent in an external circuit, through which the solar cell delivers its power. The semiconductor can absorb only those photons which have an energy which is greater than its band gap. The size of the semiconductor band gap thus determines the proportion of sunlight which can be converted to electrical energy. Solar cells consist normally of two absorbing materials with different band gaps in order to very effectively utilize the solar energy. Most organic semiconductors have exciton diffusion lengths of up to 10 nm. There is still a need here for organic semiconductors through which the excited state can be passed on over very large distances. It has now been found that, surprisingly, the compounds of the general formula (I) described above are particularly advantageously suitable for use in excitonic solar cells.

Suitable organic solar cells generally have a layer structure and generally comprise at least the following layers: anode, photoactive layer and cathode. These layers generally consist of a substrate customary therefore. The structure of organic solar cells is described, for example, in US 2005/0098726 A1 and US 2005/0224905 A1, which are fully incorporated here by reference.

Suitable substrates are, for example, oxidic materials (such as glass, ceramic, SiO₂, especially quartz, etc.), polymers (e.g. polyvinyl chloride, polyolefins such as polyethylene and polypropylene, polyesters, fluoropolymers, polyamides, polyurethanes, polyalkyl (meth)acrylates, polystyrene and mixtures and composites thereof) and combinations thereof.

Suitable electrodes (cathode, anode) are in principle metals (preferably of groups 2, 8, 9, 10, 11 or 13 of the Periodic Table, e.g. Pt, Au, Ag, Cu, Al, In, Mg, Ca), semiconductors (e.g. doped Si, doped Ge, indium tin oxide (ITO), gallium indium tin oxide (GITO), zinc indium tin oxide (ZITO), etc.), metal alloys (e.g. based on Pt, Au, Ag, Cu, etc., especially Mg/Ag alloys), semiconductor alloys, etc. The anode used is preferably a material essentially transparent to incident light. This includes, for example, ITO, doped ITO, ZnO, TiO₂, Ag, Au, Pt. The cathode used is preferably a material which essentially reflects the incident light. This includes, for example, metal films, for example of Al, Ag, Au, In, Mg, Mg/Al, Ca, etc.

For its part, the photoactive layer comprises at least one or consists of at least one layer which comprises, as an organic semiconductor material, at least one compound which is selected from compounds of the formula (I) as defined above. In one embodiment, the photoactive layer comprises at least one organic acceptor material. In addition to the photoactive layer, there may be one or more further layers, for example a layer with electron-conducting properties (ETL, electron transport layer) and a layer which comprises a hole-conducting material (hole transport layer, HTL) which need not absorb, exciton- and hole-blocking layers (e.g. EBLs) which should not absorb, multiplication layers. Suitable exciton- and hole-blocking layers are described, for example, in U.S. Pat. No. 6,451,415.

Suitable exciton blocker layers are, for example, bathocuproins (BCPs), 4,4′,4″-tris[3-methylphenyl(phenyl)amino]triphenylamine (m-MTDATA) or polyethylenedioxythiophene (PEDOT), as described in U.S. Pat. No. 7,026,041.

The inventive excitonic solar cells are based on photoactive donor-acceptor heterojunctions. When at least one compound of the formula (I) is used as the HTM (hole transport material), the corresponding ETM (exciton transport material) must be selected such that, after excitation of the compounds, a rapid electron transfer to the ETM takes place. Suitable ETMs are, for example, C60 and other fullerenes, perylene-3,4:9,10-bis(dicarboximides) (PTCDs), etc. When at least one compound of the formula (I) is used as the ETM, the complementary HTM must be selected such that, after excitation, a rapid hole transfer to the HTM takes place. The heterojunction may have a flat configuration (cf. Two layer organic photovoltaic cell, C. W. Tang, Appl. Phys. Lett., 48 (2), 183-185 (1986) or N. Karl, A. Bauer, J. Holzäpfel, J. Marktanner, M. Möbus, F. Stölzle, Mol. Cryst. Liq. Cryst., 252, 243-258 (1994).) or be implemented as a bulk heterojunction (or interpenetrating donor-acceptor network; cf., for example, C. J. Brabec, N. S. Sariciftci, J. C. Hummelen, Adv. Funct. Mater., 11 (1), 15 (2001).). The photoactive layer based on a heterojunction between at least one compound of the formula (I) and an HTL (hole transport layer) or ETL (exciton transport layer) can be used in solar cells with MiM, pin, pn, Mip or Min structure (M=metal, p=p-doped organic or inorganic semiconductor, n=n-doped organic or inorganic semiconductor, i=intrinsically conductive system of organic layers; cf., for example, J. Drechsel et al., Org. Eletron., 5 (4), 175 (2004) or Maennig et al., Appl. Phys. A 79, 1-14 (2004)). It can also be used in tandem cells, as described by P. Peumnas, A. Yakimov, S. R. Forrest in J. Appl. Phys, 93 (7), 3693-3723 (2003) (cf. patents U.S. Pat. No. 4,461,922, U.S. Pat. No. 6,198,091 and U.S. Pat. No. 6,198,092). It can also be used in tandem cells composed of two or more MiM, pin, Mip or Min diodes stacked on one another (cf. patent application DE 103 13 232.5) (J. Drechsel et al., Thin Solid Films, 451452, 515-517 (2004)).

Thin layers of the compounds and of all other layers can be produced by vapor deposition under reduced pressure or in inert gas atmosphere, by laser ablation or by solution- or dispersion-processible methods such as spin-coating, knife-coating, casting methods, spraying, dip-coating or printing (e.g. inkjet, flexographic, offset, gravure; intaglio, nanoimprinting). The layer thicknesses of the M, n, i and p layers are typically from 10 to 1000 nm, preferably from 10 to 400 nm.

The substrates used are, for example, glass, metal foils or polymer films which are generally coated with a transparent conductive layer (for example SnO₂:F, SnO₂:In, ZnO:Al, carbon nanotubes, thin metal layers).

In addition to the compounds of the general formula (I), the following semiconductor materials are suitable for use in organic photovoltaics:

acenes such as anthracene, tetracene, pentacene and substituted acenes. Substituted acenes comprise at least one substituent selected from electron-donating substituents (e.g. alkyl, alkoxy, ester, carboxylate or thioalkoxy), electron-withdrawing substituents (e.g. halogen, nitro or cyano) and combinations thereof. These include 2,9-dialkylpentacenes and 2,10-dialkylpentacenes, 2,10-dialkoxypentacenes, 1,4,8,11-tetraalkoxypentacenes and rubrene (5,6,11,12-tetraphenylnaphthacene). Suitable substituted pentacenes are described in US 2003/0100779 and U.S. Pat. No. 6,864,396. A preferred acene is rubrene (5,6,11,12-tetraphenylnaphthacene).

Phthalocyanines, such as hexadecachlorophthalocyanines and hexadecafluorophthalocyanines, metal-free phthalocyanine and phthalocyanine comprising divalent metals, especially those of titanyloxy, vanadyloxy, iron, copper, zinc, especially copper phthalocyanine, zinc phthalocyanine and metal-free phthalocyanine, copper hexadecachlorophthalocyanine, zinc hexadecachlorophthalocyanine, metal-free hexadecachlorophthalocyanine, copper hexadecafluorophthalocyanine, hexadecafluorophthalocyanine or metal-free hexadecafluorophthalocyanine.

Porphyrins, for example 5, 10,15,20-tetra(3-pyridyl)porphyrin (TpyP).

Liquid-crystalline (LC) materials, for example hexabenzocoronene (HBC-PhC12) or other coronenes, coronenediimides, or triphenylenes such as 2,3,6,7,10,11-hexahexylthiotriphenylene (HTT6) or 2,3,6,7,10,11-hexakis(4-n-nonylphenyl)triphenylene (PTP9), 2,3,6,7,10,11-hexakis(undecyloxy)triphenylene (HAT11). Particular preference is given to LCs which are discotic.

Thiophenes, oligothiophenes and substituted derivatives thereof. Suitable oligothiophenes are quaterthiophenes, quinquethiophenes, sexithiophenes, α,ω-di(C₁-C₈)alkyloligothiophenes such as α,ω-dihexylquaterthiophenes, α,ω-dihexylquinquethiophenes and α,ω-dihexylsexithiophenes, poly(alkylthiophenes) such as poly(3-hexylthiophene), bis(dithienothiophenes), anthradithiophenes and dialkylanthradithiophenes such as dihexylanthradithiophene, phenylene-thiophene (P-T) oligomers and derivatives thereof, especially α,ω-alkyl-substituted phenylene-thiophene oligomers.

Preferred thiophenes, oligothiophenes and substituted derivatives thereof, are poly-3-hexylthiophene (P3HT) or compounds of the α,α′-bis(2,2-dicyanovinyl)quinquethiophene (DCV5T) type, poly(3-(4-octylphenyl)-2,2′-bithiophene) (PTOPT), poly(3-(4′-(1″,4″,7″-trioxaoctyl)phenyl)thiophene) (PEOPT), poly(3-(2′-methoxy-5′-octylphenyl)thiophenes) (POMeOPTs), poly(3-octylthiophene) (P3OT), pyridine-containing polymers such as poly(pyridopyrazine vinylene), poly(pyridopyrazine vinylene) modified with alkyl groups e.g. EHH-PpyPz, PTPTB copolymers, polybenzimidazobenzophenanthroline (BBL), poly(9,9-dioctylfluorene-co-bis-N,N′-(4-methoxyphenyl)-bis-N,N′-phenyl-1,4-phenylenediamine) (PFMO); see Brabec C., Adv. Mater., 2996, 18, 2884. (PCPDTBT) poly[2,6-(4,4-bis(2-ethylhexyl)-4H-cyclopenta[2,1-b;3,4-b′]-dithiophene)-4,7-(2,1,3-benzothiadiazoles)].

Paraphenylenevinylene and paraphenylenevinylene-comprising oligomers and polymers, for example polyparaphenylenevinylene (PPV), MEH-PPV (poly(2-methoxy-5-(2′-ethylhexyloxy)-1,4-phenylenevinylene)), MDMO-PPV (poly(2-methoxy-5-(3′,7′-dimethyloctyloxy)-1,4-phenylenevinylene)), cyano-paraphenylenevinylene (CN-PPV), CN-PPV modified with alkoxy groups.

PPE-PPV hybrid polymers (phenylene-ethynylene/phenylene-vinylene hybrid polymers).

Polyfluorenes and alternating polyfluorene copolymers, for example with 4,7-dithien-2′-yl-2,1,3-benzothiadiazoles, and also poly(9,9′-dioctylfluorene-co-benzothiadiazole) (F₈BT), poly(9,9′-dioctylfluorene-co-bis-N,N′-(4-butylphenyl)-bis-N,N′-phenyl-1,4-phenylenediamine) (PFB).

Polycarbazoles, i.e. carbazole-comprising oligomers and polymers, such as (2,7) and (3,6).

Polyanilines, i.e. aniline-comprising oligomers and polymers.

Triarylamines, polytriarylamines, polycyclopentadienes, polypyrroles, polyfuran, polysilols, polyphospholes, N,N′-Bis-(3-methylphenyl)-N,N′-bis-(phenyl)-benzidine (TPD), 4,4′-bis(carbazol-9-yl) biphenyl (CBP), 2,2′,7,7′-tetrakis-(N,N-di-p-methoxyphenyl-amine)-9,9′-spirobifluorene (spiro-MeOTAD).

Fullerenes, especially C60 and derivatives thereof such as PCBM (=[6,6]-phenyl-C₆₁-butyric acid methyl ester). In such cases, the fullerene derivative would be a hole conductor.

Copper(I) iodide, copper(I) thiocyanate.

p-n-Mixed materials, i.e. donor and acceptor in one material, polymer, block copolymers, polymers with C60s, C60 azo dyes, trimeric mixed material which comprises compounds of the carotenoid type, porphyrin type and quinoid liquid-crystalline compounds as donor/acceptor systems, as described by Kelly in S. Adv. Mater. 2006, 18, 1754.

All aforementioned semiconductor materials may also be doped. Examples of dopants: Br₂, tetrafluorotetracyanoquinodimethane (F₄-TCNQ), etc.

The invention further provides an organic light-emitting diode (OLED) which comprises at least one compound of the general formula (I) as defined above. The compounds of the formula (I) may serve as a charge transport material (electron conductor).

Organic light-emitting diodes are in principle constructed from several layers. These include 1. anode 2. hole-transporting layer 3. light-emitting layer 4. electron-transporting layer 5. cathode. It is also possible that the organic light-emitting diode does not have all of the layers mentioned; for example, an organic light-emitting diode with the layers (1) (anode), (3) (light-emitting layer) and (5) (cathode) is likewise suitable, in which case the functions of the layers (2) (hole-transporting layer) and (4) (electron-transporting layer) are assumed by the adjacent layers. OLEDs which have the layers (1), (2), (3) and (5) or the layers (1), (3), (4) and (5) are likewise suitable. The structure of organic light-emitting diodes and processes for their production are known in principle to those skilled in the art, for example from WO 2005/019373. Suitable materials for the individual layers of OLEDs are disclosed, for example, in WO 00/70655. Reference is made here to the disclosure of these documents. Inventive OLEDs can be produced by methods known to those skilled in the art. In general, an OLED is produced by successive vapor deposition of the individual layers onto a suitable substrate. Suitable substrates are, for example, glass or polymer films. For vapor deposition, it is possible to use customary techniques such as thermal evaporation, chemical vapor deposition and others. In an alternative process, the organic layers may be coated from solutions or dispersions in suitable solvents, for which coating techniques known to those skilled in the art are employed. Compositions which, as well as a compound of the general formula (I) have a polymeric material in one of the layers of the OLED, preferably in the light-emitting layer, are generally applied as a layer by processing from solution.

As a result of the inventive use of the compounds (I), it is possible to obtain OLEDs with high efficiency. The inventive OLEDs can be used in all devices in which electroluminescence is useful. Suitable devices are preferably selected from stationary and mobile visual display units. Stationary visual display units are, for example, visual display units of computers, televisions, visual display units in printers, kitchen appliances and advertising panels, illuminations and information panels. Mobile visual display units are, for example, visual display units in cell phones, laptops, digital cameras, vehicles and destination displays on buses and trains. Moreover, the compounds (I) may be used in OLEDs with inverse structure. The compounds (I) in these inverse OLEDs are in turn preferably used in the light-emitting layer. The structure of inverse OLEDs and the materials typically used therein are known to those skilled in the art.

Before they are used as charge transport materials or exciton transport materials, it may be advisable to subject the compounds of the formula (I) to a purification process. Suitable purification processes comprise conversion of the compounds of the formula (I) to the gas phase. This includes purification by sublimation or PVD (physical vapor deposition). Preference is given to a fractional sublimation. For fractional sublimation and/or deposition of the compound, a temperature gradient is used. Preference is given to subliming the compound of the formula (I) with heating in a carrier gas stream. The carrier gas then flows through a separating chamber. A suitable separating chamber has at least two different separating zones with different temperatures. Preference is given to using a three-zone furnace. A suitable process and an apparatus for fractional sublimation is described in U.S. Pat. No. 4,036,594.

The invention further provides a process for depositing at least one compound of the formula (I) onto or applying at least one compound of the formula (I) to a substrate by a gas phase deposition process or a wet application process.

The invention is illustrated in detail with reference to the following nonrestrictive examples.

### EXAMPLES

### General Method for Determining the Transistor Characteristics

### Production of Semiconductor Substrates by Means of Physical Vapor Deposition (PVD)

### Device Preparation: Bottom-Gate Top-Contact Configuration

The substrates used for the devices were highly doped n-type (100 nm) silicon wafers (<0.004 Ω⁻¹cm). SiO₂ layer (unit area-based capacitance Cᵢ=10 nF/cm²) as gate dielectric were thermally grown to 3000 Å thickness onto the Si substrates. The SiO₂/Si substrates were cleaned by washing with acetone followed by isopropanol. Organic semiconductor thin films (45 nm) were vapor-deposited onto the Si/SiO₂ substrates held at well-defined temperatures between 25 and 150° C. (typically 125° C.) with a deposition rate of 0.3-0.5 Å/s at 10⁻⁶ torr, employing a vacuum deposition chamber (Angstrom Engineering, Inc., Canada). Thin film transistors in top-contact configuration were used to measure the charge mobility of the materials. Gold source and drain electrodes (typical channel length were 100 μm with width/length ratios of about 20) were vapor-deposited through a shadow mask. The current-voltage (I-V) characteristics of the devices were measured using a Keithley 4200-SCS semiconductor parameter analyzer. Key device parameters, such as charge carrier mobility (μ) and on-to-off current ratio (Iₒₙ/I_{off}) were extracted from the source-drain current (I_{d}) vs. gate voltage (V_{g}) characteristics employing standard procedures.

### Surface Treatment

Subsequently, the surfaces of the substrates are modified by treatment with n-octadecyltriethoxysilane (OTS, C₁₈H₃₇Si(OC₂H₅)₃), obtained from Aldrich Chem. Co.). To this end, a few drops of OTS were loaded on top of a preheated quartz block (about 100° C.) inside a vacuum desiccator. The desiccator was immediately evacuated under vacuum (about 25 mm Hg) for one minute and the valve to vacuum was closed. The SiO₂/Si substrate was treated to give a hydrophobic surface for at least 5 hours. Subsequently, the substrates were baked at 110° C. for 15 minutes, rinsed with isopropanol and dried with a stream of nitrogen.

### Example 1

### N,N′-bis(Heptafluorobutyl)perylene-3,4:9,10-tetracarboxylic diimide (PBI)

1.0 g (2.54 mmol) of perylene-3,4:9,10-tetracarboxylic bisanhydride are dissolved in 15 ml of dry N-methylpyrrolidone (NMP) and treated with ultrasound for 30 minutes. 1.43 g (7.19 mmol) of 2,2,3,3,4,4,4-heptafluorobutylamine and 920 mg of acetic acid are then added. The mixture is stirred at 200° C. in a pressure vessel for 12 hours and then poured onto 100 ml of 2N HCl. The solid formed is filtered off and dried. The crude product is purified by column chromatography with dichloromethane to obtain a red powder; yield: 482 mg (25%). ¹H NMR (CDCl₃): δ 8.78 (d, ³J=8.0 Hz, 2H), 8.71 (d, ³J=8.1 Hz, 2H), 5.04 (t, ³J=15.5 Hz, 4H); ¹⁹F NMR (376.49 MHz, CDCl₃); δ −80.97 (t, J=9.8 Hz, 6H), −116.39 (m, 4H), −128.22 (m, 4H); melting point: 421° C.; HR-MS (APCI (neg. mode, chloroform, acetonitrile)): 789.0264 (M+Cl⁻), calculated 789.0268 (C₃₂H₁₂F₁₄N₂O₄Cl); UV/Vis (CH₂Cl₂): λ max (∈)=524 (85 200), 488 (50 900), 457 nm (18 500 M⁻cm⁻¹); cyclic voltammetry (CH₂Cl₂, 0.1M tetrabutylammonium hexafluorophosphate (TBAHFP), vs. ferrocene): E^{red} ½ (PBI/PBI⁻)=−0.95 V, E^{red} ½ (PBI⁻/PBI²⁻)=−1.15.V.

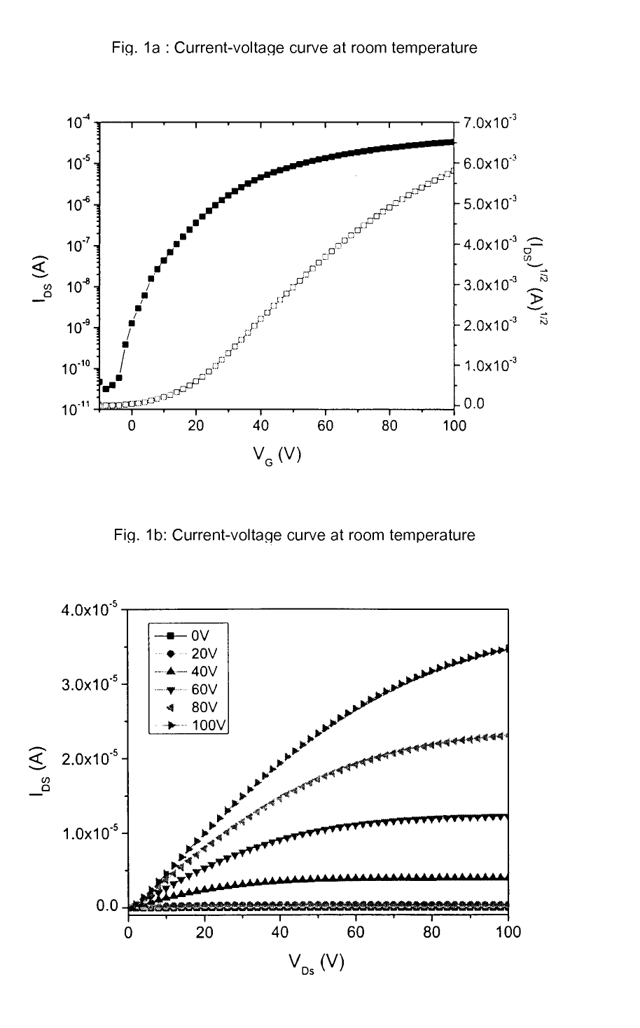

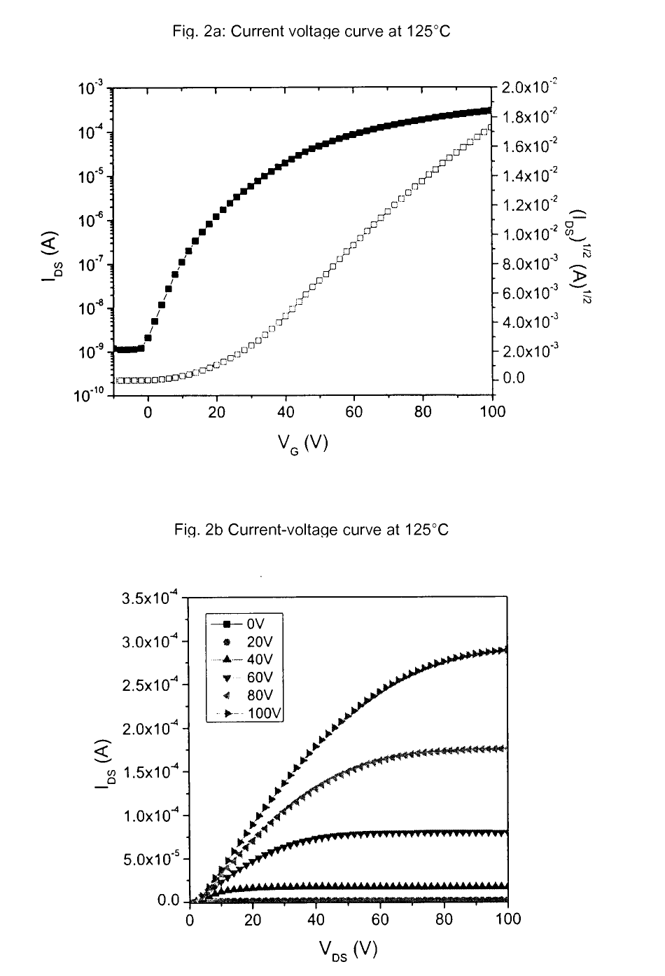

The compound was purified by sublimation three times in a three-zone sublimation apparatus (Lindberg/Blue Thermo Electron Corporation, high vacuum 4.6×10⁻⁴ Torr). The three temperature zones were operated at 250° C., 190° C. and 148° C. To produce semiconductor substrates, the material from temperature zone 2 was used. Semiconductor substrates according to the general method for the PVD process are used. The results are shown in FIGS. 1 and 2.

| | | Substrate temperature | |
|---|---|---|---|
| | | Room | |
| | | temperature | 125° C. |
| handled only under | Mobility (cm²/Vs) | 0.061 | 0.735 |
| protective gas | | | |
| handled in the atmosphere | Mobility (cm²/Vs) | 0.057 | 0.561 |

### Example 2

### N,N′-Bis(2,2,3,3,4,4,5,5,5-nonafluoropentyl)-3,4:9,10-tetracarboxylic diimide

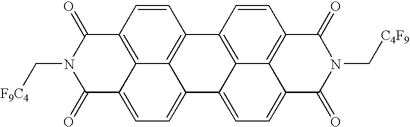

2.23 g (5.69 mmol) of perylene-3,4:9,10-tetracarboxylic bisanhydride, 4.00 g (16.1 mmol) of nonafluoropentylamine, 2.00 g of acetic acid in 34 ml of dry NMP are heated at 200° C. in a pressure vessel for 48 hours. After cooling to room temperature, the mixture is poured onto 2N HCl and the solid formed is filtered off. The solid is repeatedly heated in an aqueous solution of sodium hydrogen carbonate (2% strength solution) to remove remaining bisanhydride. The solid is filtered off and crystallized from toluene to give 655 mg (0.767 mmol, 13% of theory) of the title compound.

¹H-NMR (400 MHz, CDCl₃, TMS): δ=5.05 (t, 4H, ³J (H,F)=15.8 Hz), 8.72 (d, 4H, ³J (H,H)=8.1 Hz), 8.78 (d, 4H, ³J (H,H)=8.0 Hz);

HR-MS (apci (neg.-mode)): 854.0526 (M⁻), calculated 854.0515 (C₃₄H₁₂F₁₈N₂O₄) electrochemistry (CH₂Cl₂, 0.1M TBAHFP, vs. ferrocene):

E^{red}_{1/2}(PBI/PBI⁻)=−0.96 V, E^{red}_{1/2}(PBI⁻/PBI²⁻)=−1.15. V.

The title compound was purified by sublimation in a three-zone sublimation apparatus (Lindberg/Blue Thermo Electron Corporation, high vacuum 4.6×10⁻⁴ Torr). The three temperature zones were operated at 300° C., 230° C. and 100° C. starting with 304.6 mg of the title compound to give: A1 (deep red): 226 mg, A2 (red): 9.6 mg and residue (dark brown) 12 mg.

To produce semiconductor substrates, the material from temperature zone 2 was used. Semiconductor substrates according to the general method for the PVD process are used.

| | | Measurement atmosphere | | |
|---|---|---|---|---|
| | | N₂ | | Air*⁾ |
| Substrate | | Temperature (° C.) | | |
| treatment | | 25 | 125 | 125 |
| with OTS | Mobility μ (cm²/Vs) | 0.044 | 0.11 | 0.096 |
| | | (0.040) | (0.11) | (0.092) |
| | Iₒₙ/I_{off} | 1.4 × 10⁸ | 3.0 × 10⁶ | 2.8 × 10⁶ |
| | Vₜ (V) | 27.3 | 26.6 | 29.4 |
| without OTS | Mobility μ (cm²/Vs) | — | 0.067 | 0.026 |
| | | | (0.060) | (0.025) |
| | Iₒₙ/I_{off} | — | 2.0 × 10⁴ | 1.9 × 10² |
| | Vₜ (V) | — | 12.6 | 15.7 |

*⁾relative humidity 50%
**⁾Parenthesis: μ from the slope of V_{GS} vs (I_{Ds})^{1/2}

The device was subjected to an annealing process at 150° C. for 60 min under nitrogen. After said annealing, the device shows the following characteristics:

μ: 0.61 cm²/Vs

### Vₜ: 36.9 V

Iₒₙ/I_{off}: 5.7×10⁶

1-12. (canceled)

13. A charge transport material comprising a compound of formula I 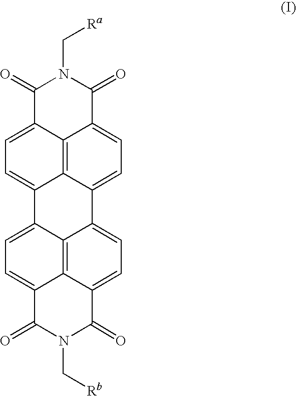
wherein
R^{a} and R^{b} are each independently perfluoro-C₂-C₄-alkyl.

14. The charge transport material according to claim 13, wherein R^{a} and R^{b} are each n-heptafluoropropyl.

15. A method of conducting elections in the presence of the charge transport material according to claim 13, wherein the conducting occurs in an organic field-effect transistor, or an organic solar cell, or in an organic light-emitting diode.

16. A semiconductor material in an organic electronic, comprising the charge transport material according to claim 13.

17. An n-semiconductor in an organic field-effect transistor comprising the semiconductor material according to claim 16.

18. An active material comprising the charge transport material according to claim 13, wherein the compound of Formula I is present as an active material in an organic photovoltaic cell.

19. A fluorescent dye comprising the charge transport material according to claim 13, wherein the compound of Formula I is present as a fluorescent dye in a display based on fluorescence conversion; in a light-collecting plastics part which is optionally combined with a solar cell; as a pigment dye in an electrophoretic display; or as a fluorescent dye in an application based on chemoluminescence.

20. An organic field-effect transistor comprising a substrate having at least one gate structure, a source electrode and a drain electrode and at least one compound of formula I as defined in claim 13 as an n-semiconductor.

21. A substrate comprising a plurality of organic field-effect transistors, wherein at least one of the organic field-effect transistors comprises the compound of formula I as defined in claim 13.

22. A semiconductor unit comprising at least one substrate as defined in claim 21.

23. An organic light-emitting diode (OLED) comprising at least one compound of formula I as defined in claim 13.

24. A process of forming a substrate, comprising
depositing at least one compound of formula I 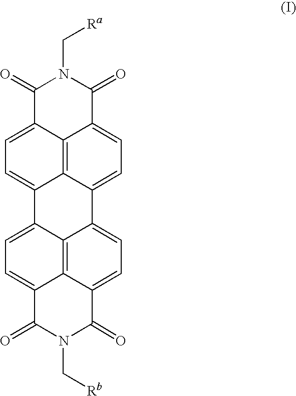
wherein
R^{a} and R^{b} are each independently perfluoro-C₂-C₄-alkyl, on, or applying at least one compound of the formula I to, a substrate by a gas phase deposition process or a wet application process.

25. An organic field-effect transistor comprising a substrate having at least one gate structure, a source electrode and a drain electrode and at least one compound of formula I, as defined in claim 14, as an n-semiconductor.

26. A substrate comprising a plurality of organic field-effect transistors, wherein at least one of the organic field-effect transistors comprises the compound of formula I as defined in claim 14.

27. An exciton transport material comprising a compound of formula I 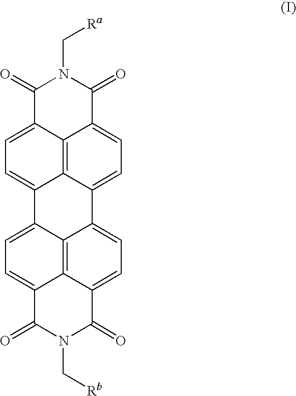
wherein
R^{a} and R^{b} are each independently perfluoro-C₂-C₄-alkyl.

28. The exciton transport material according to claim 26, wherein R^{a} and R^{b} are each n-heptafluoropropyl.

29. A semiconductor material in an organic electronic, comprising the exciton transport material according to claim 27.

30. An active material comprising the exciton transport material according to claim 27, wherein the compound of Formula I is present as an active material in an excitonic solar cell.

31. An organic light-emitting diode (OLED) comprising at least one compound of formula I as defined in claim 27.
